(19) United States
(12) Patent Application Publication
(10) Pub. No.: US 2025/0009367 A1
(43) Pub. Date: Jan. 9, 2025

(54) PATELLA CLAMP AND PATELLA TRACKING SYSTEM

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Timothy J. Petteys, Bartlett, TN (US); Roger Ryan Dees, JR., Drummonds, TN (US); Randy C. Winebarger, Southaven, MS (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/705,662

(22) PCT Filed: Dec. 6, 2022
(86) PCT No.: PCT/US2022/051949

§ 371 Date: Apr. 29, 2024

Related U.S. Application Data
(60) Provisional application No. 63/287,662, filed on Dec. 9, 2021.

(51) Int. Cl. A61B 17/17; A61B 17/15; A61B 34/10; A61B 34/20

(52) U.S. Cl. CPC A61B 17/1767; A61B 17/158; A61B 34/10; A61B 34/20; A61B 2034/104; A61B 2034/105; A61B 2034/2046

(57) ABSTRACT

Disclosed herein is a patella clamp configured to grip a patient's patella. The patella clamp includes a clamping assembly, a ratchet arm assembly, and a handle assembly. The clamping assembly includes first and second patella grip portions mounted in opposing relationship. In use, actuation of the handle assembly moves the second patella grip portion towards the first patella grip portion to clamp the patient's patella. The handle assembly is configured to be moveably adjustable (e.g., rotatable) relative to the clamping assembly. In addition, and/or alternatively, the patella clamp may be configured as a tracking patella clamp arranged and configured to be used in an orthopedic computer-assisted and/or robotic surgical surgery to enable a surgeon to view a virtual patella model to visualize patella preparation steps in a computer-based environment prior to making the preparations on the patient.

### CROSS-REFERENCE TO RELATED APPLICATION

This application claims the benefit of U.S. Provisional Patent Application Ser. No. 63/287,662, filed Dec. 9, 2021, and titled “Patella Clamp and Patella Tracking System,” the entire contents of which application is hereby incorporated by reference in its entirety.

### TECHNICAL FIELD

The present disclosure is directed to an orthopedic surgical instrument, and, more specifically, to a patella clamp that may include a tracking element for use in a computer-assisted and/or robotic surgical system to facilitate determining characteristics of the patella for surgical planning and preparing the patella during a surgical procedure.

### BACKGROUND

Orthopedic patella clamps are known. Generally speaking, a patella clamp includes a handle assembly and a clamping, gripping, or resection assembly (terms used interchangeably herein without the intent to limit). In use, the clamping assembly may include first and second grip portions for engaging a patient's patella positioned therebetween. In one example, the first and second grip portions may engage the patient's patella in the superior/inferior direction and the handle assembly may include, for example, a threaded shaft and nut for moving the first and second grip portions. Alternatively, the first and second grip portions may engage the patient's patella in the medial/lateral direction and the handle assembly may include, for example, a pistol grip.

In either event, one disadvantage of known patella clamps is that the clamping assembly and the handle assembly remain in the same position with respect to each other. That is, the handle assembly is not adjustable relative to the clamping assembly. Having the position of the handle assembly fixed with respect to the clamping assembly limits the opportunity of use by a surgeon.

Another disadvantage of known patella clamps and patella preparation processes is that surgeons cannot see the results of any resection prior to making the cuts with the patella clamp. As a result, when utilizing existing methods such as free-hand cutting of the patient's patella, using resection cutting guides, reaming, or the like, surgeons are not always confident of the plane they may be creating.

It would be beneficial to provide a lightweight adjustable patella clamp that allows increased visibility when resecting a patient's patella. For example, by arranging the patella clamp so that the clamping assembly and the handle assembly can be moveably adjustable relative to each other, the patella clamp may accommodate variations in subluxation or degrees of eversion surgical techniques as well as left and right knees and medial versus lateral approaches.

In addition, and/or alternatively, it would be beneficial to provide a patella clamp arranged and configured to be used in a computer-assisted and/or robotic surgical system to enable modeling, visualization, and/or the like of the patella and/or resulting preparation steps (for instance, resection, reaming, cuts, and/or the like) to the patella.

Accordingly, there remains a need for an improved patella clamp. It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is a patella clamp including a clamping assembly and a handle assembly. In use, the clamping assembly is arranged and configured to grip a patient's patella. In accordance with one or more features of the present disclosure, the handle assembly is arranged and configured to be moveably adjustable relative to the clamping assembly. For example, in any preceding or subsequent example, the clamping assembly is coupled to the handle assembly via a multi-positional ratcheting assembly (e.g., an adjustable ratchet assembly) so that the handle assembly can be rotated relative to the clamping assembly. Thus arranged, in use, the patella clamp is arranged and configured to enable the surgeon to reposition (e.g., move, rotate, etc.) the handle assembly out of the way to enable, for example, increased visibility and/or to accommodate other anatomy.

Disclosed herein is a surgical system configured to generate a virtual patella model of a patient patella. The surgical system may include or may use a tracking patella clamp. The surgical system may include a tracking system communicatively coupled to at least one tracker coupled to the tracking patella clamp. The tracking patella clamp may include a clamping assembly and a handle assembly. In use, the clamping assembly is arranged and configured to grip the patient's patella. The surgical system may execute a computer-assisted surgical application operative to generate patella information based on tracking information received from the at least one tracker. The patella information may be associated with at least one patella characteristic, such as location, dimensions, cutting planes, and/or the like. The patella model may be presented via a display device and configured to be a virtual representation of the patient's patella. In any preceding or subsequent example, the at least one tracker may include a tracking array of optical sensors for use with an optical tracking system.

In any preceding or subsequent example of the surgical system, a user may input a patella preparation step. Non-limiting examples of a patella preparation step may include a cut, a resection, burring, and/or the like. The computer-assisted surgical system may present the patella model with the patella preparation step to facilitate user visualization of a patella preparation step prior to physically performing the step on the actual patient patella. In any preceding or subsequent example, the computer-assisted surgical system may present performance information associated with the patella preparation step. A non-limiting example, of performance information may include range-of-motion information.

In any preceding or subsequent example, a tracking patella clamp may include a clamping assembly operative to engage a patella, a handle assembly coupled to the clamping assembly, in which the handle assembly is operative to actuate to secure the patella within the clamping assembly, and at least one tracking element operative to determine tracking information of the patella.

In any preceding or subsequent example, the handle assembly may be operative to rotate relative to the clamping assembly. In any preceding or subsequent example, the at least one tracking element may be coupled to the clamping assembly.

In any preceding or subsequent example, the tracking information may include at least one of position information or orientation information of the patella. In any preceding or subsequent example, the tracking information may include a cutting plane associated with the patella.

In any preceding or subsequent example, the at least one tracking element may be configured to determine instrument information of a surgical instrument associated with the tracking patella clamp. In any preceding or subsequent example, the instrument information may include a location of the surgical instrument relative to the patella. In any preceding or subsequent example, the at least one tracking element may be configured to determine the instrument information via detecting a reflective surface of the surgical instrument. In any preceding or subsequent example, the surgical instrument may include a reamer tool.

In any preceding or subsequent example, a computer-assisted surgical system may include at least one computing device, comprising a display device, processing circuitry, and a memory coupled to the processing circuitry. The memory may include instructions that, when executed by the processing circuitry, cause the processing circuitry to receive tracking information associated with a patella detected by at least one tracking element coupled to a tracking patella clamp engaging the patella, determine patella information based on the tracking information, the patella information indicating at least one characteristic of the patella, generate a patella model configured as a virtual representation of the patella configured based on the patella information, and present a graphical user interface (GUI) object visually depicting the patella model on a GUI screen via the display device.

In any preceding or subsequent example, the instructions, when executed by the processing circuitry, may cause the processing circuitry to receive instrument tracking information corresponding to a surgical instrument associated with the tracking patella clamp, in which the instrument tracking information may be detected by the at least one tracking element.

In any preceding or subsequent example, the instructions, when executed by the processing circuitry, may cause the processing circuitry to determine instrument information of the surgical instrument based on the instrument tracking information, in which the instrument information may include at least one of a position or an orientation of the surgical instrument.

In any preceding or subsequent example, the instructions, when executed by the processing circuitry, may cause the processing circuitry to present a GUI model of the surgical instrument on the GUI screen based on the instrument information.

In any preceding or subsequent example, the surgical instrument may include a reamer tool. In any preceding or subsequent example, the tracking information may include a cutting plane associated with the patella.

Preceding or subsequent examples of the present disclosure provide numerous advantages. In one non-limiting example advantage, the clamping assembly (e.g., first and second patella grip portions) of a patella clamp may be rotated relative to a handle assembly. This allows for the handle assembly to be moved out of the way during examination, evaluation, and/or resection of the patella. Thus arranged, increased visibility may be provided (e.g., a surgeon can see the patella better). In addition, an increased working window can be created enabling a surgeon to utilize a resection tool and/or a reaming guide without interference from the handle assembly. Furthermore, the patella may be gripped with the patella in a wider variety of positions.

Preceding or subsequent examples of the present disclosure provide numerous technological advantages and technical features over conventional systems. One non-limiting example of a technological advantage may include providing a tracking patella clamp of the present disclosure that may be used in connection with a computer-assisted and/or robotic surgical system. Another non-limiting example of a technological advantage may include providing a surgical system configured to allow a surgeon to visualize a patella preparation step and/or performance information associated with a patella preparation step prior to making any adjustments to the actual patient patella. Surgical planning processes according to any preceding or subsequent example may, among other things, facilitate planning of patella preparation and/or implant device positioning with decreased complexity and improved efficiency and effectiveness as compared to conventional systems. For example, surgical planning processes according to any preceding or subsequent example may allow healthcare professionals to avoid invalid patella preparation steps and/or avoid erroneous or ineffective patella preparations during the planning and/or performance of a surgical procedure, saving time and effort and improving patient outcomes. Preceding or subsequent examples are not limited in this context. Additional technological advantages and technical features over conventional systems would be known to those of skill in the art based on the present disclosure.

Preceding or subsequent examples described in the present disclosure may provide improvements in computing technology. For example, managing the complexity of planning the placement and/or preparation of a patella has long been problematic for healthcare professionals using conventional computer-assisted tools, particularly managing changes during a surgical procedure. The preparation of a patella involves multiple parameters (e.g., depth, angles, and/or the like) as well as interactions with other portions of patient anatomy. These parameters lead to a multidimensionally complex configuration space which the surgeon (or other healthcare professional) has to explore while trying to achieve the goals of the surgery, for instance, retaining or regaining proper kinematics of a knee joint.

Computer-assisted surgical planning processes according to any preceding or subsequent example may provide improvements in computing technology through, for example and without limitation, determining a patella model that may facilitate easier and more efficient evaluation of patella preparations, for example, allowing for virtual visualization prior to actual physical preparations of the patella, which are not available using existing computing systems.

Surgical planning processes according to any preceding or subsequent example may be applied to various practical applications. For example, patella models may be applied by a surgeon and/or computing system to generate a surgical plan. In another example, patella models may be applied by a surgeon and/or computing system to administer surgery to a patient, such as performing an arthroplasty procedure. In an additional example, surgical planning processes (and patella models thereof) may be integrated into a surgical system hardware and/or hardware used to plan and/or perform a surgical procedure. Preceding or subsequent examples are not limited in this context. Additional practical applications would be known to those of skill in the art based on the present disclosure.

Further features and advantages of at least some of the preceding or subsequent examples of the present invention, as well as the structure and operation of various preceding or subsequent examples of the present invention, are described in detail below with reference to the accompanying drawings.

### BRIEF DESCRIPTION OF THE DRAWINGS

By way of illustration, various examples of the disclosed device will now be described, with reference to the accompanying drawings, in which:

FIG. 1 illustrates a side view of an example of a patella clamp in accordance with one or more features of the present disclosure, the patella clamp including a handle assembly, a clamping assembly, and a ratchet arm assembly;

FIG. 2 illustrates an exploded perspective view of the patella clamp shown in FIG. 1;

FIG. 3 illustrates a top view of the clamping assembly engaging a patient's patella;

FIG. 4 illustrates a side view of the ratchet arm assembly;

FIG. 5 illustrates a side view of the patella clamp shown in FIG. 1 with the clamping assembly rotated 90 degrees from horizontal;

FIG. 6 illustrates an end-view of the patella clamp shown in FIG. 1 with the clamping assembly rotated 45 degrees from horizontal;

FIG. 7 illustrates a perspective view of an example of a tracking patella clamp in accordance with one or more features of the present disclosure;

FIGS. 8A-C illustrates various views of the tracking patella clamp shown in FIG. 7 engaging a patella in accordance with one or more features of the present disclosure;

FIG. 9 is a diagram illustrating an environment for operating a system for planning and performing a joint replacement surgery in accordance with one or more features of the present disclosure;

FIG. 10 illustrates exemplary tracking arrays for use with a computer-assisted and/or robotic surgery system in accordance with one or more features of the present disclosure;

FIG. 11 is a block diagram depicting a system for performing a surgery planning process in accordance with one or more features of the present disclosure;

FIG. 12 depicts an illustrative graphical user interface for performing a surgery planning process in accordance with one or more features of the present disclosure; and

FIGS. 13A-C illustrate various views of an example of a tracking patella clamp with a patella preparation element in accordance with one or more features of the present disclosure.

It should be understood that the drawings are not necessarily to scale and that the disclosed examples are sometimes illustrated diagrammatically and in partial views. In certain instances, details which are not necessary for an understanding of the disclosed methods and devices or which render other details difficult to perceive may have been omitted. It should be further understood that this disclosure is not limited to the particular examples illustrated herein. In the drawings, like numbers refer to like elements throughout unless otherwise noted.

### DETAILED DESCRIPTION

Various features or the like of a patella clamp arranged and configured to be used within an orthopedic computer-assisted and/or robotic surgical system will now be described more fully hereinafter with reference to the accompanying drawings, in which one or more features of the patella clamp will be shown and described. It should be appreciated that the various features may be used independently of, or in combination, with each other. It will be appreciated that a patella clamp as disclosed herein may be embodied in many different forms and should not be construed as being limited to the examples set forth herein. Rather, these examples are provided so that this disclosure will convey certain features of the patella clamp to those skilled in the art.

As will be described herein, in accordance with one or more features of the present disclosure, the patella clamp includes a clamping, gripping, or resecting assembly (terms used interchangeably without the intent to limit) arranged and configured to grip a patient's patella and a handle assembly, a trigger, an arm assembly, a pistol grip, etc. (terms used interchangeably herein without the intent to limit) arranged and configured to adjust, move, etc. the clamping assembly about the patient's patella. In use, the clamping assembly is arranged and configured to be moveable adjustable relative to the handle assembly. For instance, in any preceding or subsequent example, the clamping assembly may be arranged and configured to rotate about a longitudinal axis so that, in use, the handle assembly can be rotated to provide increased visibility and/or an increased working space.

In addition, and/or alternatively, in accordance with one or more features of the present disclosure, the patella clamp may include one or more tracking elements operative to enable the patella clamp to be used with a computer-assisted and/or robotic surgical system to, among other things, enable modeling, visualization, and/or the like of the patella and/or resulting preparation steps to the patella.

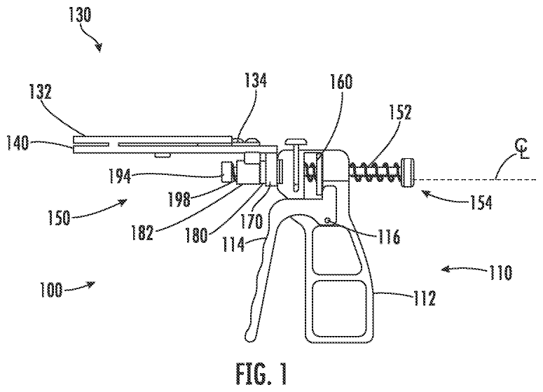

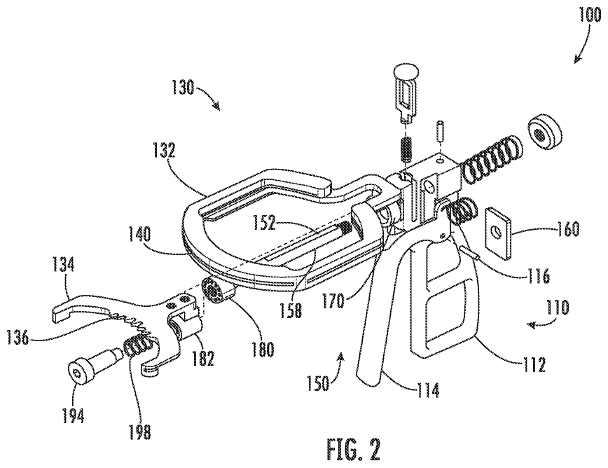

Referring to FIGS. 1 and 2, an example of a patella clamp 100 in accordance with one or more features of the present disclosure will now be shown and described. As shown, the patella clamp 100 includes a handle assembly 110 and a clamping assembly 130. As illustrated, in any preceding or subsequent example, the handle assembly 110 include a first arm 112 and a second arm 114. In any preceding or subsequent example, the second arm 114 may be moveable and the first arm 112 may be stationary, although other configurations are envisioned such as, for example, where both the first and second arms 112, 114 may be moveable relative to each other. In any preceding or subsequent example, the second arm 114 may be coupled to the first arm 112 via a pivot pin 116. As will be readily appreciated by one of ordinary skill in the art, thus arranged, in use, a user squeezes the second arm 114 towards the first arm 112 to move, adjust, and/or clamp the clamping assembly 130 about the patient's patella as will be described in greater detail below. As such, as illustrated, the handle assembly 110 may be in the form of a pistol grip, although the handle assembly 110 may be provided in any other suitable mechanisms now known or hereafter developed. As such, the present disclosure should not be limited to any particular type of handle assembly unless explicitly claimed.

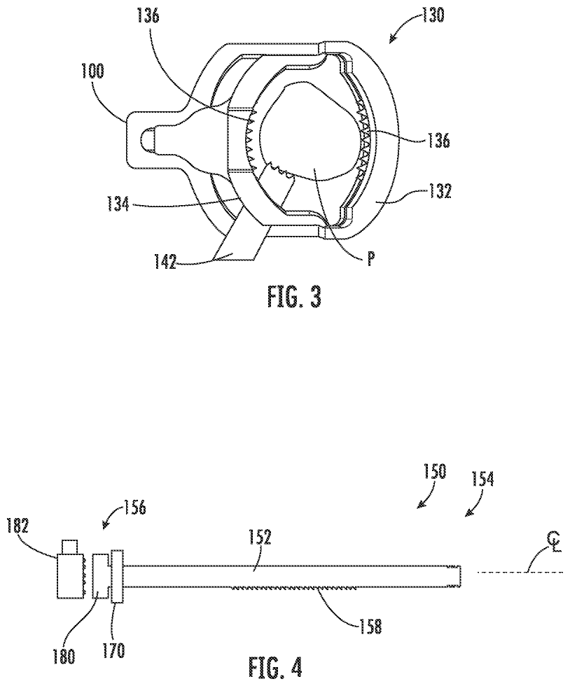

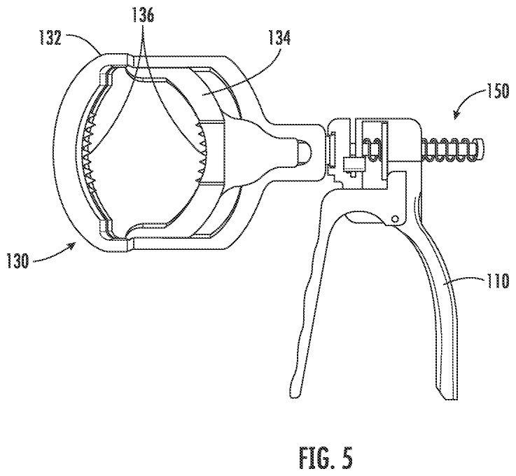

As illustrated, in any preceding or subsequent example, the clamping assembly 130 includes a first patella grip portion 132 and a second patella grip portion 134. In any preceding or subsequent example, the first and second patella grip portions 132, 134 may be arranged and configured in opposing mating relationship with the second patella grip portion 134 moveably positioned relative to the first patella grip portion 132. For example, the second patella grip portion 134 may be arranged and configured to move towards the first patella grip portion 132 via actuation of the handle assembly 110. Thus arranged, with additional reference to FIG. 3, a user can move the second patella grip portion 134 towards the first patella grip portion 132 to capture a patient's patella P positioned therebetween. In addition, in any preceding or subsequent example, as best illustrated in FIGS. 3 and 5, the first and second patella grip portions 132, 134 may include teeth, spikes, serrations, etc. 136 (terms used interchangeably herein without the intent to limit) to capture or grab the patient's patella P. In any preceding or subsequent example, the first and second patella grip portions 132, 134 may be arranged and configured to provide an opening (e.g., distance between the first and second patella grip portions 132, 134) of approximately 60 mm to grip various sized patellas, although this is but one configuration and other dimensions may be used.

As such, with reference to FIG. 3, in use, the first patella grip portion 132 can hold the patient's patella P using the teeth 136 associated therewith. Thereafter, by actuating the handle assembly 110, the second patella grip portion 134 can be moved towards the patient's patella P to engage the patient's patella P with opposing teeth 136 associated with the second patella grip portion 134. As illustrated, in any preceding or subsequent example, a sawblade 142 may be inserted from the side of first patella grip portion 132. For example, the sawblade 142 may enter through dedicated cutting slots or may rest on a surface of the first patella grip portion 132. In use, the sawblade 142 may be used to cut or resect a portion of the patient's patella P (e.g., cutting posterior surface of the patient's patella P).

As illustrated, in any preceding or subsequent example, the handle assembly 110 may be coupled to the clamping assembly 130 by a ratchet arm assembly 150, although it is envisioned that the handle assembly 110 may be coupled to the clamping assembly 130 by other suitable mechanisms now known or hereafter developed. In use, the ratchet arm assembly 150 is arranged and configured to transfer actuation of the handle assembly 110 to the clamping assembly 130 so that, as will be appreciated by one of ordinary skill in the art, actuation of the handle assembly 110 (e.g., user squeezes the first and second arms 112, 114 so that the second arm 114 moves toward the first arm 112), the second patella grip portion 134 is moved towards the first patella grip portion 132 to, for example, engage a patient's patella P positioned therebetween.

In any preceding or subsequent example, with reference to FIG. 4, the ratchet arm assembly 150 includes a ratchet arm 152 having a first end 154, a second end 156, and a plurality of teeth 158 positioned between the first and second ends 154, 156. The ratchet arm assembly 150 may also include a lever 160 (FIGS. 1 and 2) and a bushing 170. In use, the lever 160 is mounted over the ratchet arm 152 in contact with the teeth 158 formed on the ratchet arm 152. In use, as will be readily appreciated by one of ordinary skill in the art, activation of the handle assembly 110 pushes on a lower portion of lever 160 towards the second end 156 of the ratchet arm 152. The lever 160 sequentially engages and/or interacts with the plurality of teeth 158 on the ratchet arm 152 to translate the ratchet arm 152 distally in the direction of the second end 156.

With reference to FIGS. 1, 2, and 4, the ratchet arm 152 includes an engagement element (e.g., a first engagement element) 180 coupled to or associated with the second end 156 thereof. In use, the engagement element 180 is non-rotatably coupled to the ratchet arm 152 so that the engagement element 180 does not rotate about a longitudinal axis C_{L} of the ratchet arm 152. Similarly, as illustrated, the second patella grip portion 134 includes an engagement element (e.g., a second engagement element) 182. In use, the first and second engagement elements 180, 182 include mating surfaces having mating features. In use, the mating features on the mating surface of the second engagement element 182 associated with the second patella grip portion 134 is arranged and configured to couple with the mating features on the mating surface of the first engagement element 180 of the ratchet arm 152. For example, in any preceding or subsequent example, the mating features may be provided as a plurality of corresponding raised features and recessed features arranged and configured to mate with each other (e.g., a plurality of mating male and female protrusions and recesses). For example, the raised features may be semi-spherical in nature, although the exact shape and number of features is not crucial and thus the raised features may be provided in alternate forms such as, but not limited to, mating teeth or the like. Additionally, mating features are not limited to raised features and recessed features and may be provided in any other suitable form now known or hereafter developed including, for example, a Hirth coupling, ball plungers, a splined coupling, or any other type of mechanical linkage or arrangement that allows for selective engagement and rotation of the first and second engagement elements.

For example, in any preceding or subsequent example, the mating surface of the first engagement element 180 may include a series of detents arranged in a radial pattern. The mating features on the mating surface of the second engagement element 182 may include a series of protrusions arranged in a radial pattern. Alternatively, the detents may be on the second engagement element 182 and the protrusions may be on the first engagement element 180. In use, the protrusions selectively mate with the series of detents to secure a position of the second engagement element 182, and hence the second patella grip portion 134, relative to the first engagement element 180 and hence the ratchet arm 152 and the handle assembly 110.

In any preceding or subsequent example, the first engagement element 180 associated with the ratchet arm 152 may include an internally threaded bore to mate with a fastener 194 (FIGS. 1 and 2). The second engagement element 182 associated with the second patella grip portion 134 includes a through-hole arranged and configured to allow the fastener 194 to pass therethrough so that the fastener 194 may threadably engage the threaded bore 192 of the first engagement element 180 of the ratchet arm 152.

As illustrated, in any preceding or subsequent example, as previously mentioned, the ratchet arm assembly 150 may also include a bushing 170. In use, the bushing 170 is mounted over the ratchet arm 152 (e.g., the bushing 170 includes an opening arranged and configured to allow the ratchet arm 152 to pass therethrough). In use, the bushing 170 is free to rotate about the longitudinal axis C_{L} of the ratchet arm 152. In addition, the bushing 170 is free to translate relative to the ratchet arm 152. In use, the bushing 170 is coupled to or associated with the first patella grip portion 132. As such, in use, the bushing 170 couples the first patella grip portion 132 to the ratchet arm 152. Thus arranged, in use, actuation of the handle assembly 110 cause the first engagement element 180 and the second engagement element 182 to move distally in the direction of the second end 156 of the ratchet arm 152. In addition, since the second patella grip portion 134 is coupled to the second engagement element 182, the second patella grip portion 134 moves distally. However, because the first patella grip portion 132 is coupled to the ratchet arm assembly 150 via the bushing 170, which is free to rotate and translate along the ratchet arm 152, the first patella grip portion 132 remains stationary and does not move relative to ratchet arm 152.

As best illustrated in FIG. 2, the ratchet arm assembly 150 may also include a first biasing element 198 positioned about the fastener 194 between an enlarged head portion of the fastener 194 and the second engagement element 182 associated with the second patella grip portion 134. In this particular example, the fastener 194 retains the first biasing element 198 and the second engagement element 182 on the second end 156 of the ratchet arm 152. Thus arranged, the first biasing element 198, which may be in the form of a coiled spring, biases the second engagement element 182 associated with the second patella grip portion 134 against the first engagement element 180 associated with the ratchet arm 152. In use, a user may grip, hold, etc. the first engagement element 180 and pull on the second engagement element 182 against the bias of the first biasing element 198 so that the mating features on the first and second engagement elements 180, 182 are decoupled or separated (e.g., move the second engagement 182 away from the first engagement element 180 and the handle assembly 110). Thereafter, with the second engagement element 182 separated from the first engagement element 180, the handle assembly 110 can be rotated relative to the first and second patella grip portions 132, 134 (e.g., the clamping assembly 130) about the longitudinal axis C_{L} of the ratchet arm 152. Once the desired orientation of the first and second patella grip portions 132, 134 (e.g., the clamping assembly 130) is reached, a user may release the second engagement element 182 causing the first biasing element 198 to bias the second engagement element 182 into engagement with the first engagement 180 (e.g., the second engagement element 182 and the first engagement element 180 are biased back together in mating relationship). Thus arranged, with reference to FIG. 5, in accordance with features of the present disclosure, the clamping assembly 130 (e.g., the first and second patella grip portions 132, 134) can be rotated relative to the handle assembly 110. For example, as illustrated, the clamping assembly 130 (e.g., the first and second patella grip portions 132, 134) can be oriented at about 90 degrees from horizontal.

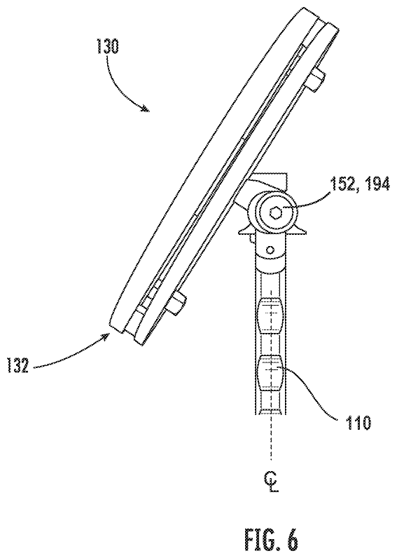

In addition, with reference to FIG. 6, by disengaging the second engagement element 182 from the first engagement element 180, the clamping assembly 130 (e.g., the first and second patella grip portions 132, 134) can be rotated, angulated, etc. relative to the longitudinal axis C_{L} of the ratchet arm 152. As illustrated, for example, the clamping assembly 130 (e.g., the first and second patella grip portions 132, 134) can be oriented at about 45 degrees from longitudinal axis C_{L} of the ratchet arm 152.

Referring to FIGS. 1 and 2, in any preceding or subsequent example, the first patella grip portion 132 may include a cutting slot 140. As illustrated, the cutting slot 140 may extend circumferentially about the first patella grip portion 132 (e.g., the cutting slot 140 may extend around a perimeter of first patella grip portion 132). For example, the cutting slot 140 may extend approximately 270 degrees, although this is but one example and the cutting slot 140 may extend more or less. In use, the cutting slot 140 is arranged and configured to receive a sawblade 142 (FIG. 4) to resect a portion of the patent's patella.

Additional description and features of the patella clamp 100 can be found in United States Patent Application Publication No. 2021/0378683, filed on Jun. 3, 2021, entitled “Patella Clamp,” the entire contents of which are hereby incorporated by reference in their entirety.

In any preceding or subsequent example, methods, systems, apparatuses, devices, and/or combinations thereof may provide a surgical planning process for a computer-assisted and/or robotic surgery system (or computer assisted surgery system (CASS)). The surgical planning process may include generating a surgical plan for at least a portion of an implant surgery. In any preceding or subsequent example, the portion of the implant surgery may be or may include a patella preparation process and/or implant installation procedure. In any preceding or subsequent example, the patella preparation process may include associating the patella with one or more tracking elements configured to provide patella information to the computer-assisted and/or robotic surgery system (and/or a computing device associated with the computer-assisted and/or robotic surgery system). The patella information may include, without limitation, an orientation, a position, dimensions, and/or the like.

In any preceding or subsequent example, the patella preparation process may include generating a visual representation of the patella based on the patella information. Non-limiting examples of the visual representation may include a graphical model, including two-dimensional (2D) and/or three-dimensional (3D) graphical user interface (GUI) objects that may be displayed via a computing device display.

In general, surgeons are unaware of the results of a patella preparation step (for instance, cutting plane of the patella) they perform today, which are typically performed free-hand or using resection instruments. However, surgeons are not always confident of the plane or other preparations they are creating. Accordingly, patella preparation processes and corresponding clamps with computer-assisted and/or robotics capability will allow surgeons to see the result prior to making any cuts or other patella preparations.

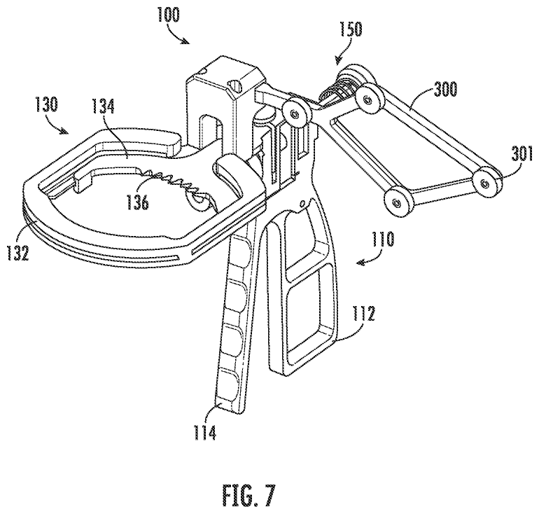

In accordance with one or more features of the present disclosure, referring to FIG. 7, the patella clamp 100 may be in the form of a “tracking patella clamp” that includes one or more tracking frames 300. In any preceding or subsequent example, the tracking frame 300 may include a plurality or array of tracking elements 301 of devices, sensors, and/or the like operative to provide for tracking, navigation, determination of orientation, determination of position, and/or the like of a portion of the patient (for instance, a patella) in a computer-assisted and/or robotic surgical system (see, for example, FIG. 14). In any preceding or subsequent example, the tracking elements 301 may be or may include fiducial markers, markers, trackers, sensors, and/or the like (terms used interchangeably herein without the intent to limit). In any preceding or subsequent example, tracking frame 300 and/or tracking elements 301 may be, may include, and/or may be coupled to one or more transceivers (for instance, a wireless transmitting device) configured to transmit information detected by tracking frame 300 and/or tracking elements 301, for example, to a computing device.

In any preceding or subsequent example, tracking frame 300 and/or tracking elements 301 may be coupled to clamping assembly 130 and, as a result, may be stationary with respect to the patella. In any preceding or subsequent example, tracking frame 300 and/or tracking elements 301 may be coupled to handle assembly 110 and, as a result, may be configured to rotate with handle assembly 110. In such a “rotating” example, a tracking element may be associated with clamping assembly 130 and/or patella to provide a reference (or “triangulate”) for tracking frame 300 and/or tracking elements 301 so that the tracking information may be collected and may be correct in any possible rotational orientation. In another “rotating” example, an offset (for instance, based on the position of handle assembly 110) may be determined in order to format the tracking information to correspond to the actual position of tracking frame 300 and/or tracking elements 301.

A tracking patella clamp configured according to any preceding or subsequent example may allow a surgeon the ability to use reamers, saw blades, and/or other preparation tools in combination with computer-assisted and/or robotic surgery techniques. For example, with a tracker attached to the resection jaw of a tracking patella clamp, the surgeon may be able to visualize the planar cut on a patella (and/or other patella preparation) and interaction with femoral (and/or other anatomy and/or components, such as an implant component) prior to making any cuts or other preparation steps.

In any preceding or subsequent example, markers 301 be coupled to various parts of patella clamp 100. For example, markers 301 may be dimensionally linked to a front jaw section (for instance, first patella grip portion 132) since it will be fixed and the back jaw (for instance, second patella grip portion 134) translates. Markers 301 (and/or additional markers not shown in FIG. 7) may be attached to any portion of patella clamp, including, without limitation, first patella grip portion 132, second patella grip portion 134, handle assembly, and/or the like.

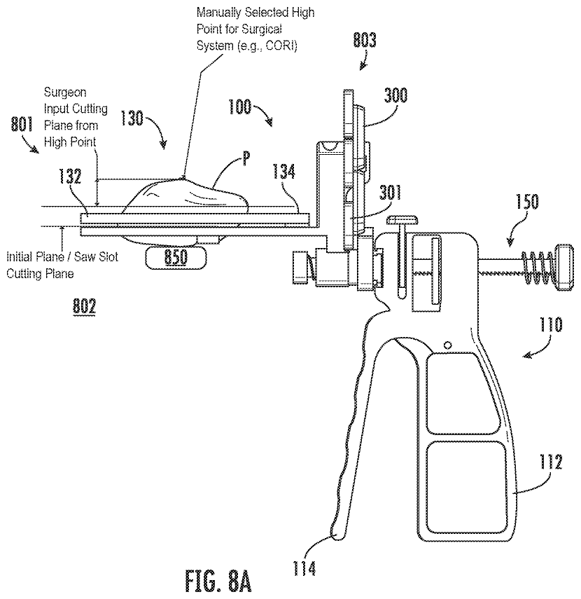

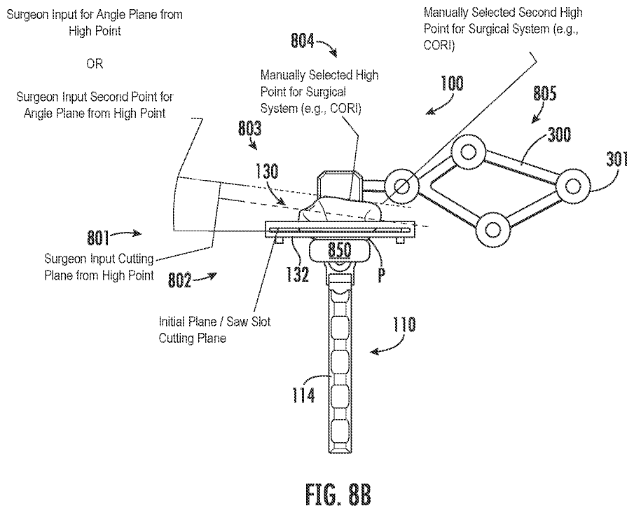

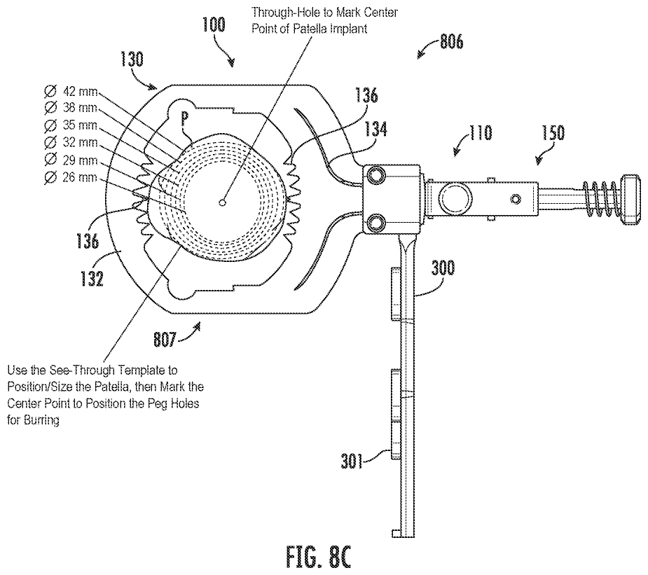

FIGS. 8A-8C illustrate various views of the example of a tracking patella clamp 100 depicted in FIG. 7 engaged with a patella P, more specifically, a side view, a front view, and a top view, respectively, of the patella clamp 100. Referring to FIG. 8A, therein is provided patella preparation processes and/or configurations associated with the patella clamp 100, including a high point cutting plane 801, an initial or saw slot cutting plane 802, and a manually selected high point (for instance, for a robotic surgery system). Referring to FIG. 8B, therein is depicted patella preparation processes and/or configurations associated with patella clamp 100 of an input for angle plane from high point or input second point for angle plane from high point 804 and a manually selected second high point for an angle plane 805. Referring to FIG. 8C, therein is depicted patella preparation processes and/or configurations associated with patella clamp 100 of a through hole 806, for example, to mark a center point of a patella implant. In any preceding or subsequent example, a see-through template 807 may be used to position/size the patella P to mark the center point for a robotic surgical system, for instance, for burring or other preparation processes.

In any preceding or subsequent example, tracking patella clamp 100 may allow for the determination, tracking, monitoring, and/or the like of characteristics of a patella. Non-limiting examples of patella characteristics may include location, orientation, dimensions (e.g., thickness), preparation elements (e.g., reamed hole depth, circumference, and/or the like), and/or the like.

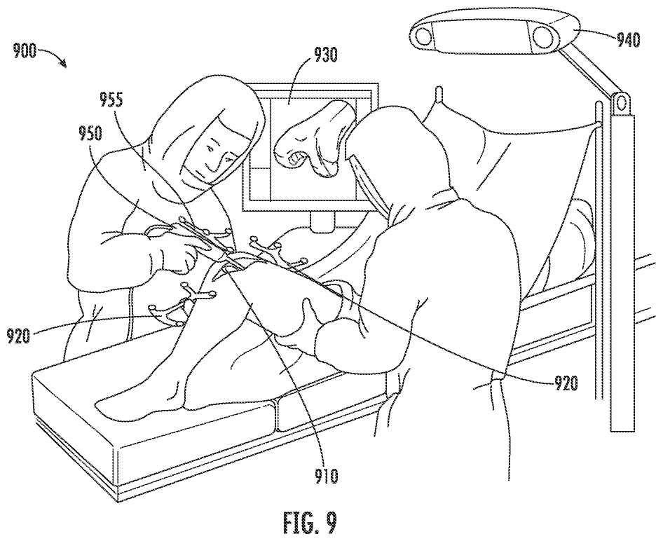

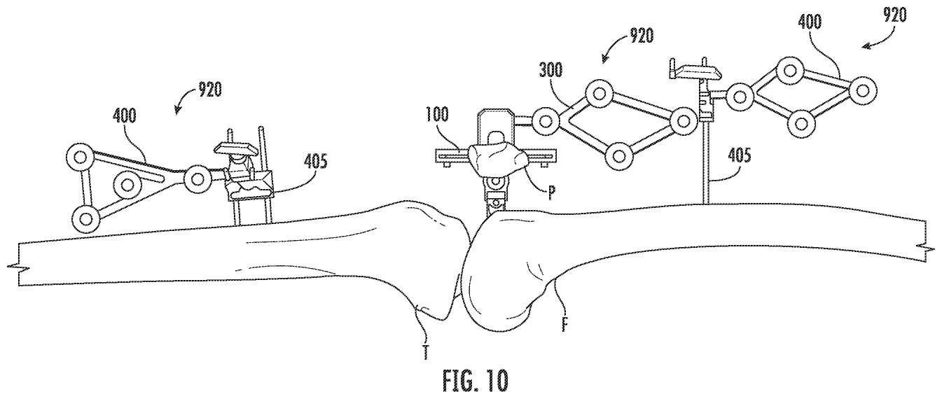

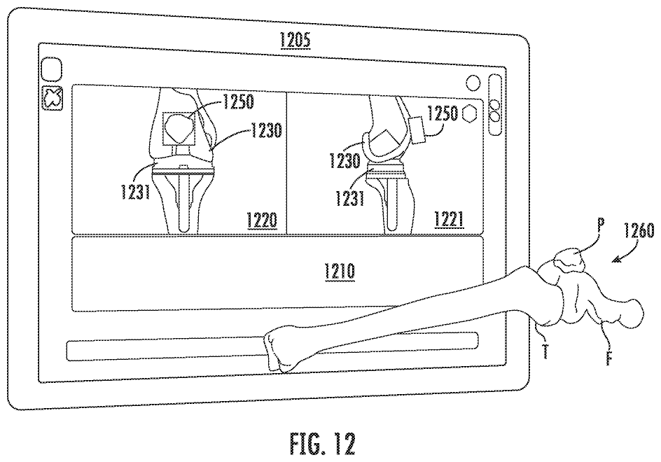

In any preceding or subsequent example, tracking elements 301 may allow for tracking, such as 3D tracking, compared to markers placed on other portions of the patient, such as the femur (see, for example, FIG. 10). Elements 301 may be tracked by an optical tracking unit. A computing device (see, for example, FIG. 9) may be able to produce a simulated patella (a “patella model”) by probing anatomic features of the patella (e.g., width, height, thickness, medial facet thickness, lateral facet thickness, etc.) (see, for example, FIG. 12). These probed regions may be linked to areas previously probed on the patient, such as the femur. Markers 301 and associated patient information may be used to tell surgeons or other users information regarding post-operative patellafemoral tracking.

Referring to FIG. 9, therein is depicted an illustration of a system 900 for performing a surgical procedure using a computer-assisted and/or robotic system. In any preceding or subsequent example, system 900 may be or may include an image-free (for instance, CT-less) system. In other examples, system 900 may be or may include an image-based system based on diagnostic image data. In any preceding or subsequent example, system 900 may operate using a combination of image-free and image-based processes. Preceding or subsequent examples are not limited in this context.

System 900 may include a surgical cutting tool 950 with an associated tracking frame 955 (also referred to as a tracking array 955), graphical user interface 930, a tracking system 940, and patient tracking frames 920 (for instance, one or more trackers, tracking arrays, and/or the like). The illustration also includes an incision 910 through which, for example, a knee revision surgery may be performed.

In an example, the illustrated robotic surgical system 900 depicts a hand-held computer-controlled surgical robotic system, for instance, the same or similar to the Cori® and/or Navio® Surgical System from Smith & Nephew, Inc. of Cordova, Tennessee, United States. System 900 may use a tracking system 940, or other type of tracking system, coupled to a robotic controller to track and control a hand-held surgical instrument. For example, tracking system 940 tracks tracking array 955 coupled to surgical tool 950 and tracking arrays 920 coupled to the patient to track locations of the instrument relative to the target bone (see, for example, FIG. 9).

Referring to FIG. 10, therein are depicted illustrative tracking arrays 920 for use with the computer-assisted and/or robotic system 900 of FIG. 9. As shown in FIG. 10, tracking arrays 920 may include tracking arrays 400 coupled to patient bone via posts or mounts 405 to track locations of an instrument or surgical device relative to the target bone, for instance, a femur F and/or tibia T for a knee procedure. In addition, a tracking array 300 may be associated with the patella P via patella clamp 100.

Trackers 301 and/or 401 may be or may include any type of device, sensor, communication element, and/or the like capable of interfacing with a tracking system of a computer-assisted and/or robotic surgical system (such as tracking system 940 of FIG. 9) to provide location, orientation, and/or the like information associated with patient anatomy to a computer-assisted and/or robotic surgical system. Illustrative and non-limiting examples of trackers 301 and/or 401 may include infrared sources, Radio Frequency (RF) sources, ultrasound sources, wireless transmitters, and/or the like. In any preceding or subsequent example, trackers 301 may include infrared trackers configured to be used with an optical tracking system. Preceding or subsequent examples are not limited in this context.

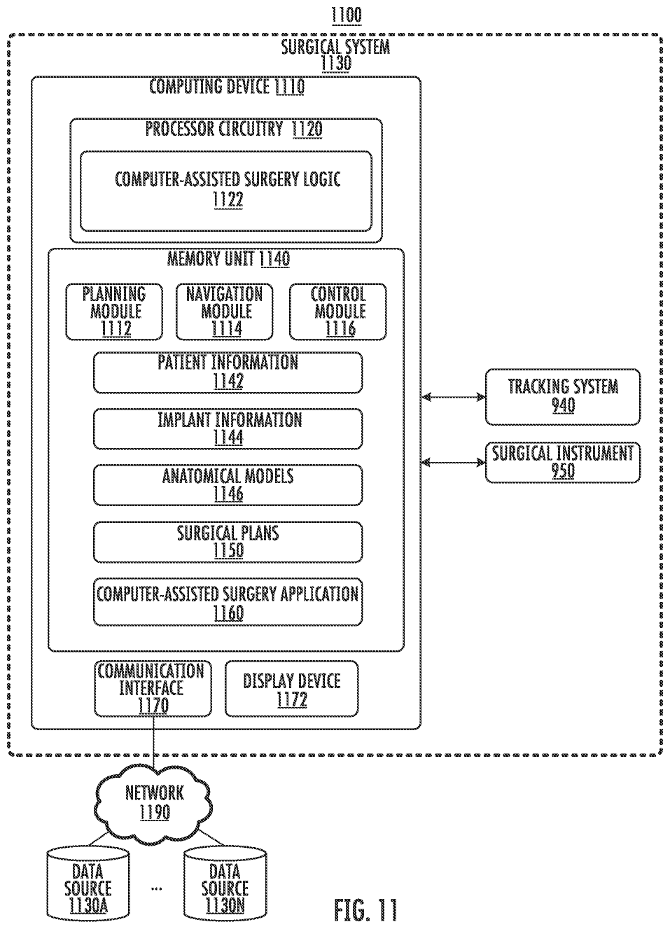

FIG. 11 is a block diagram depicting an operating environment 1100 for planning and/or performing a surgical procedure according to an example. A non-limiting example of a surgical procedure may include an orthopedic or arthroplasty procedure.

As shown in FIG. 11, operating environment 1100 may include a surgical system 1130. In any preceding or subsequent example, surgical system 1130 may include a computing device 1110 communicatively coupled to network 1190 via a communication interface (for instance, a transceiver) 1160. Computing device 1110 may be or may include one or more logic devices, including, without limitation, a server computer, a client computing device, a personal computer (PC), a workstation, a laptop, a notebook computer, a smart phone, a tablet computing device, and/or the like. Although a single computing device 1110 is depicted in FIG. 11, preceding or subsequent examples are not so limited, as surgical system 1130 may include and/or operably communicate with, and surgery planning processes according to any preceding or subsequent example may be performed using, a plurality of computing devices 1110. In addition, components of computing device 1110 depicted in FIG. 11 may be arranged within a plurality of different computing devices. In any preceding or subsequent example, surgical system 1130 may be or may be a part of system 900 of FIG. 9. Preceding or subsequent examples are not limited in this context.

In any preceding or subsequent example, surgical system 1130 may include computing device 1110 operating as or operating as part of a control system (for instance, system 900), a tracking system 940, a surgical instrument 950, and/or the like. Optionally, in any preceding or subsequent example, surgical system 1130 may also include or may be operatively coupled to a display device 1172 and/or a one or more data sources (for instance, databases) 1130a-n. In any preceding or subsequent example, display device 1172 and/or databases 1130a-n may be used to provide navigation and control of a surgical instrument 1150, which may include navigation and control of a cutting tool, a point probe, a bur, a bone saw, a reamer, or other tools/instruments, that may be used during an arthroplasty procedure, such as an orthopedic (or similar) prosthetic implant surgery.

Control system 1110 may include one or more computing devices configured to coordinate information received from tracking system 930 and provide control to surgical instrument 950. In any preceding or subsequent example, control system 1110 may include a planning module 1112, a navigation module 1114, a control module 1116, and a communication interface 1170. In any preceding or subsequent example, planning module 1112 may provide pre-operative planning services that allow surgeons or other healthcare professionals to virtually plan a procedure prior to reshaping the patella (and other portions of the knee joint) during an implant procedure on the patient.

In an example, such as for a total knee arthroplasty (TKA) procedure, planning module 1112 may be used to generate and/or present a virtual model of a patella (“patella model”). The patella model may be manipulated in reference to a virtual implant host model (for instance, a “knee joint model”) and/or virtual implant device (“implant model”) (see, for example, FIG. 12).

In any preceding or subsequent example, the virtual model of the implant host (for instance, the joint to be revised, including the patella, and/or components thereof) may be created through use of a point probe or similar instrument tracked by optical tracking system 940. Control system 1110, for instance, via planning module 1112, may be used to collect data (patella information) from surfaces of the patella to create a patella model in the form of a virtual model of the patient's actual anatomical structure. This method may, among other things, increase the accuracy of the planning process by using data collected from the patient's actual patella to allow for, among other things, the accurate and/or iterative reshaping of the patella to ensure proper fit of new prosthetic implants and optimization of anatomical alignment. The patella model may be generated based on the patella information, including, without limitation, width, height, maximum thickness, medial facet thickness, lateral facet thickness, bone quality, highest point, lowest point, and/or the like.

In an example, navigation module 1114 may coordinate tracking the location and orientation of the implant, the implant host (for instance, the patient patella, femur, tibia, and/or the like), and/or surgical instrument 950. In certain examples, navigation module 1114 may also coordinate tracking of the virtual models (for instance, a patella model) used during pre-operative or intra-operative planning within planning module 1112. Tracking the virtual models may include operations such as alignment of the virtual models with the implant host, generating predicted performance information, through data obtained via tracking system 940. In these examples, navigation module 1114 receives input from tracking system 940 regarding the physical location and orientation of surgical instrument 150 and the patella of the patient. Tracking of the implant host may include tracking multiple individual bone structures, such as with tracking arrays 300 and/or 400. For example, during a TKA procedure, tracking system 940 may track the patella using tracking device 300 engaged with the patella (for example, as illustrated in FIG. 10).

In an example, control module 1116 may process information provided by navigation module 1114 to generate control signals for displaying patella information on display 1172 and/or controlling surgical instrument 950. In certain examples, control module 1116 may also work with navigation module 1114 to produce visual animations to assist the surgeon during an operative procedure. Visual animations may be displayed via a display device, such as display device 1172 (see, for example, FIG. 12). In an example, the visual animations may include real-time or substantially real-time 3D representations of the implant, the patella, the femur, the tibia, and/or surgical instrument 950, among other things.

FIG. 12 depicts an illustrative graphical user interface for performing a surgery planning process in accordance with one or more features of the present disclosure. As shown in FIG. 12, a patella model 1250 configured as a graphical user interface (GUI) object, such as a virtual (for instance, virtual 3D) representation of patella P of patient anatomy 1260 may be presented on GUI interface (window or screen) 1205 (for instance, via display 1172). A virtual representation of the knee implant components, such as a femur component 1230 and a tibia component 1231 may be presented in various views, such as a front view 1220 and a side view 1221. In any preceding or subsequent example, performance information 1210 may be displayed on GUI interface 1205. In general, performance information 1210 may include metrics configured to provide an indication of the performance of the implant based on configuration of implant components and/or preparation of patient anatomy. For instance, a surgeon may implement a cut or other preparation of patella model 1250 and predicted performance information 1210 may be presented.

Referring to FIGS. 8A and 8B, additional markers outside of array 300 may be used to track the patella. In any preceding or subsequent example, markers, sensors, tracking elements, and/or the like may be arranged on various areas of the patella, femur, and/or other portions of the patient. For example, an interior marker 850 may be attached to a portion of the patella to obtain information about the location of the patella, for instance, within the femoral groove. In this manner, patella-femoral interaction may be tracked and determined by surgical system 1130 for visualization using patella model 1250 on GUI interface 1205. In another example, an anterior marker or tracking element may be arranged on the anterior side of the patella for instance, to provide information for patellafemoral simulations.

In any preceding or subsequent example, markers (such as reflective markers), sensors, tracking elements (“patella tracking elements”), and/or the like may be arranged on or around the patella for use by tracking elements 301 to determine tracking information. The tracking information may be used by a computer-assisted surgery system (see, for example, FIG. 9) to determine patella information. The patella tracking elements may have known positions, locations, orientations, and/or the like.

In any preceding or subsequent example, other anatomical regions, such as the femur and tibia, may also be probed and modeled to allow for performance information 1210 in the form of range of motion simulations, for example, to give a surgeon a current state evaluation to see if correction is needed.

In any preceding or subsequent example, patella model 1250 may be generated to visualize cuts and/or other patella preparation steps. For example, a surgeon may enter a type of cut into surgery system 1130 (for instance, via data entry or selection through computer-assisted surgery application) and the results of the cut may be presented on patella model 1250. Accordingly, a surgeon may visualize the results of a patella preparation step via GUI interface 1205 prior to making the actual patella preparation step on the patient.

In one example, through the use of patella tracking using a tracking patella clamp according to any preceding or subsequent example, a surgeon may be able to track the interaction of the femur and the patella. For instance, surgical system 1130 may be able to display via GUI interface 1205 placement of the patella directly in the patella femoral groove and allow the surgeon to visualize the kinematics (performance information 1210) though patient range of motion.

In another example, the surgeon may be able to determine a characteristic (for instance, thickness) of the patella following a preparation step (for instance, burring a specified amount from a portion of the patella).

In an additional example, surgical system 1130 may facilitate the use of burring instead of the use of a saw and saw guide. For example, surgical system 1130 may continuously monitor how much bone is being taken off and/or how much is being retained. The surgeon may probe the high point of the patella and instruct surgical system 1130 how much bone to keep or to take away. In one instance, after planar resection, the peg holes for the patella could be made by a burr and/or reamer and the results visualized via patella model 1250 on GUI interface 1205.

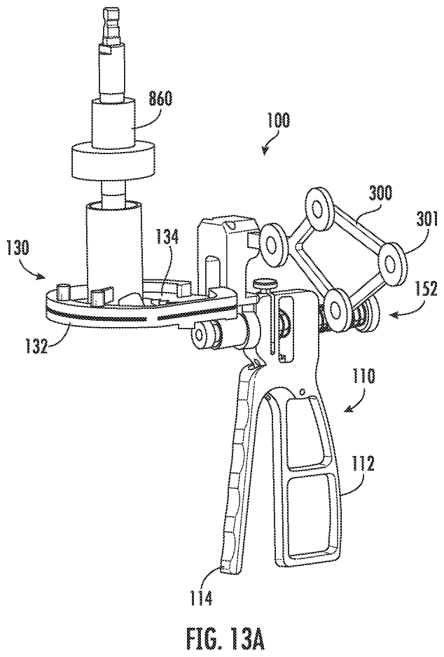

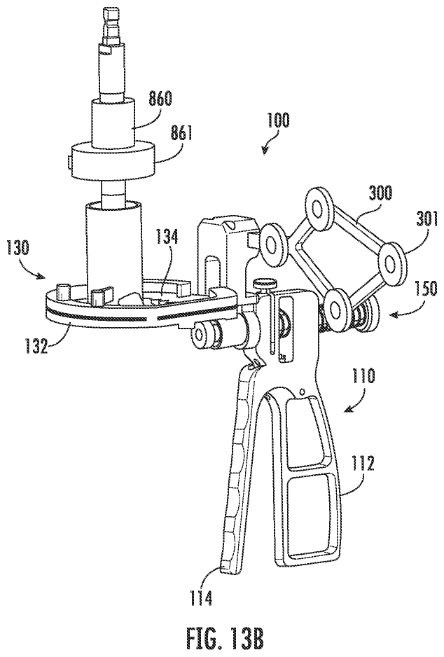

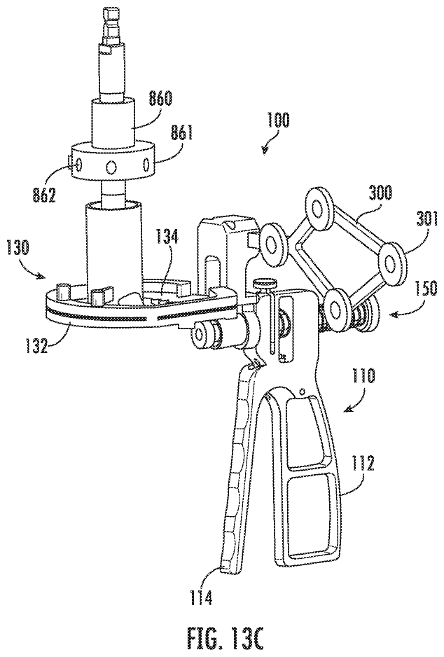

In a further example, surgical system 1130 may show the surgeon the resulting planar surface, for instance, via a collet attached to the resection guide. In this example, appropriate depth stops may be used to control depth. If a tracker was attached to the reamer or other preparation tool (see, for example, FIGS. 13B and C), depth control could be controlled via computer-assisted surgery application 1160. For example, the difference between the marker on the resection guide and the reamer could tell the reamed depth.

In another example, if a surgeon specifies a biconvex patella implant, the biconvex surface and patella peg hole could be burred automatically via a computer-assisted and/or robotic surgical system.

Referring back to FIG. 11, in any preceding or subsequent example, communication interface 1170 may facilitate communication between control system 1110 and external systems and devices. Communication interface 1170 may include both wired and wireless communication interfaces, such as Ethernet, IEEE 802.11 wireless, or Bluetooth, among others. As illustrated in FIG. 11, in this example, the primary external systems connected via the communication interface 1170 may include a tracking system 940 (which may be or may include patella tracking functionality according to any preceding or subsequent example) and a surgical instrument 950. Although not shown, databases 1130a-n and the display device 130, among other devices, may also be connected to control system 1110 via communication interface 1170. In an example, communication interface 1170 communicates over an internal bus to other modules and hardware systems within control system 1110.

In any preceding or subsequent example, tracking system 940 may provide location and orientation information for surgical devices and parts of an implant host's anatomy, including a patella registered within surgical system 1130 according to any preceding or subsequent example, to assist in navigation and control of semi-active robotic surgical devices. One illustrative and non-limiting example tracking system 940 may be the OPTOTRAK® 3-D motion and position measurement and tracking system described herein, although those of ordinary skill in the art will recognize that other tracking systems of other accuracies and/or resolutions may be used.

In an example, a surgeon may use surgical instrument 950 to shape the patella to accommodate a prosthetic implant. For instance, control surgical system 1130 may track and control a surgical cutting instrument to perform precise cuts, burring, reaming, or other preparations of the patella. In any preceding or subsequent example, a surgeon may enter a preparation instruction via GUI interface (such as cut or burr X mm from the top of the patella, ream distance Y mm into location Z on patella, and/or the like) and surgical system 1130 may provide control signals to the corresponding robotic surgical tool to implement the preparation instruction on the patient patella.

In any preceding or subsequent example, control system 1110 may operate to generate and/or access virtual or anatomical models of a patient. For example, control system 1110 may operate to generate anatomical models of a patient via an anatomical model generation process the same or similar to the methods described in U.S. Patent Application Publication No. 2019/0365474, titled “Systems and Methods for Planning and Performing Image Free Implant Revision Surgery,” and/or PCT International Application No. PCT/US2020/054231, titled “Registration of Intramedullary Canal During Revision Total Knee Arthroplasty,” both of which are incorporated by reference in the present disclosure as if fully written herein. Preceding or subsequent examples are not limited in this context. For example, surgical planning processes according to any preceding or subsequent example may generate and/or access anatomical models created via various other processes (for instance, image-based processes) capable of operating with any preceding or subsequent example described in the present disclosure.

The anatomical models may be or may include virtual representations of portions of the patient, such as patella, for example, the same or similar to the computer models of patient anatomy provided in the Cori® and/or Navio® surgical systems. The anatomical models may be graphically depicted via display device 1172 and visually manipulated (for instance, rotated, moved, viewed wholly or partially transparent or semi-transparent, viewed as wireframe or similar images, and/or the like).

In one non-limiting example, the anatomical model generation process may include an image-free process using a point probe (for instance, an optically tracked point probe) to map the actual surface of the target bone(s) that need a new implant. Mapping may be performed after removal of the defective or worn-out implant, as well as after removal of any diseased or otherwise unwanted bone. Points may be collected on the bone surfaces via “painting” by brushing or scraping the entirety of the remaining bone with the tip of the point probe. The collected points may be used to create a three-dimensional model or surface map of the bone surfaces in the computerized planning system. In another non-limiting example, an anatomical model may be mathematically accomplished by capturing a series of Cartesian coordinates that represent the tissue surface, for instance, to generate a model file (for instance, without limitation, *.stl, *.obj., *.fea, *.stp, *.sur, *.igs, *.wrl, *.xyz, and/or the like file formats). In an additional non-limiting example, the anatomical model generation process may include an image-based process based on diagnostic images of the subject anatomy of a patient, such as X-ray images, CT images, and/or the like. Image analysis software may be used to analyze the diagnostic images to generate anatomical models, such as 3D models. In any preceding or subsequent example, the anatomical model generation process may use a combination of image-free and image-based processes. In any preceding or subsequent example, at least a portion of the information used to generate a patella model may include information manually entered by a surgeon (for instance, dimensions). In general, anatomical models according to any preceding or subsequent example may be generated using various processes, including, without limitation, traditional probe painting, 3D imaging mapped with references, visual edge detection, combinations thereof, and/or the like.

For a particular surgical procedure, such as a TKA procedure, the procedure or episode of care can include three phases: pre-operative, intra-operative, and post-operative. During each phase, data may be collected or generated that can be used to analyze the episode of care. Accordingly, display device 1172 may operate as an interactive interface that can dynamically update and display how changes to the surgical plan (for instance, visualization of cuts or other patella preparations) would impact the procedure and the final performance of the implant and/or patella.

Computing device (or control system) 1110 may include a processor circuitry 1120 that may include and/or may access various logics for performing processes according to any preceding or subsequent example, for example, a surgical planning process. For instance, processor circuitry 1120 may include and/or may access a computer-assisted surgery logic 1122 and/or an anatomical model logic 1124. Processing circuitry 1120 and/or computer-assisted surgery logic 1122, and/or portions thereof may be implemented in hardware, software, or a combination thereof. As used in this application, the terms “logic,” “component,” “layer,” “system,” “circuitry,” “decoder,” “encoder,” “control loop,” and/or “module” are intended to refer to a computer-related entity, either hardware, a combination of hardware and software, software, or software in execution, examples of which may be provided by exemplary computing architecture. For example, a logic, circuitry, or a module may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, a control loop, a computational model or application, an AI model or application, an ML model or application, a proportional-integral-derivative (PID) controller, FG circuitry, variations thereof, combinations of any of the foregoing, and/or the like.

Although computer-assisted surgery logic 1122 is depicted as being within processor circuitry 1120 in FIG. 1, any preceding or subsequent examples are not so limited. For example, computer-assisted surgery logic 1122, anatomical model logic 1124, and/or any component thereof may be located within an accelerator, a processor core, an interface, an individual processor die, a memory, a storage device, a data store, a database, implemented entirely or partially as a software application (for instance, a computer-assisted surgery application 1160), and/or the like.

Memory unit 1140 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 1140 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid-state drive (SSD), and/or the like.

Memory unit 1140 may store various types of information and/or applications for performing features and processes according to any preceding or subsequent example, for instance, a surgical planning process according to any preceding or subsequent example. For example, memory unit 1140 may store patient information 1142, implant information 1144, anatomical models 1146, surgical plans 1150, and/or computer-assisted surgery application 1160. In any preceding or subsequent example, all or some of the information depicted as being stored in memory unit 1140 may be, in whole or in part, stored in data sources 1130a-n and accessible to computing device 1110.

In any preceding or subsequent example, patient information 1142 may include information for a patient undergoing a surgical procedure being performed via surgical system. Patient information 1142 may include personal information (for instance, name, address, and/or the like), physical characteristics (for instance, height, weight, and/or the like), medical information (for instance, health history, health record identifiers, procedure information, and/or the like), and/or any other type of information that may be associated with a patient. In any preceding or subsequent example, patient information 1142 may include anatomical information of the patient, such as dimensions, bone hardness, and/or other characteristics of the patient patella.

In any preceding or subsequent example, patient information 1142 may include information associated with a population of patients. For example, the population of patients may include individuals that may be used as a comparison with a patient undergoing a procedure in order to generate anatomical models, surgical plans, performance information, and/or the like according to any preceding or subsequent example. In any preceding or subsequent example, computer-assisted surgery application 1160 may operate to suggest preparation steps (for instance, cuts, planes, burring, reaming, and/or the like) to achieve certain performance metrics (for instance, kinematics) based on population patent information 1142. For example, example, an algorithm, computational model (for instance, a machine learning (ML) and/or artificial intelligence (AI) model), and/or the like may be trained using population patient information 1142 to provide preparation recommendations based on anatomy and outcomes for similar patients.

In any preceding or subsequent example, implant information 1144 may include information of an implant associated with a surgical procedure being performed via surgical system 1130. In general, implant information 1144 may include information required for modeling an implant and/or determining a configuration of an implant on or within patient anatomy. Implant information 1144 may include information that may be related to interaction of the patient patella with the implant, for example, to determine patella preparations, performance information, and/or the like.

In any preceding or subsequent example, anatomical models 1146 may include virtual models of patient anatomy associated with a surgical procedure being performed via surgical system 1130. In any preceding or subsequent example, the virtual models may include mathematical models, graphical models, feature maps, combinations thereof, and/or the like configured to virtually represent patient anatomy within a computing system (see, for example, FIG. 4). In any preceding or subsequent example, anatomical models 1146 may be configured to represent a patient patella in accordance with the present disclosure.

In any preceding or subsequent example, a surgical plan 1150 may include instructions, method, steps, workflows, and/or the like for performing a surgical procedure. In any preceding or subsequent example, surgical system 1130 may develop a surgical plan 1150 based on anatomical models 1146 and other information specific to the patient (for instance, patient information 1142) and/or implant information 1144. Surgical plans 1150 may provide a recommended patella preparation steps and preparation configurations (for instance, cut information (location, angles, and/or the like), burring information, and/or the like).

Surgical plans 1150 may be viewed preoperatively and intraoperatively, and the surgeon can modify surgical plans 1150 preoperatively or intraoperatively. For example, a surgeon may perform a “virtual” preparation of a patella, such virtually implementing a cut on the patella via GUI interface 1205 of FIG. 12. Computer-Assisted surgery logic 1122, for instance, via computer-assisted surgery application 1160, may predict, estimate, or otherwise determine the results of the virtual preparation, such as performance metrics, fit, and/or the like. The surgeon may be presented with the results of the virtual preparation and proceed accordingly (for instance, modifying or removing the virtual preparation, performing a second virtual preparation, and/or the like).

Surgical plans 1150 may be administered, in whole or in part, manually by a surgeon, automatically (for instance, computer- or robot-assisted) via computer-controlled surgical instruments 150, and/or combinations thereof. Preceding or subsequent examples are not limited in this context.

In any preceding or subsequent example, computer-assisted surgery logic 1122 may operate to perform, implement, or otherwise provide computer-assisted surgery processes for surgical system 1130 according to any preceding or subsequent example. For example, in any preceding or subsequent example, computer-assisted surgery logic 1122 may operate to manage or control operational features of planning module 1112, navigation module 1114, control module 1116, tracking system 940, and/or surgical instruments 950 described in the present disclosure. In another example, computer-assisted surgery logic 1122 may perform, implement, or otherwise provide surgical planning processes according to any preceding or subsequent example.

In any preceding or subsequent example, computer-assisted surgery application 1160 may be or may include a software application that includes and/or operates in combination with computer-assisted surgery logic 1122 to perform features of surgical system 1130 described in the present disclosure. For example, computer-assisted surgery application 1160 may operate to present user interfaces (see, for example, FIG. 12) via display device 1172.

In any preceding or subsequent example, some or all of patient information 1142, implant information 1144, anatomical models 1146, surgical plans 1150, computer-assisted surgery logic 1122, anatomical model logic 1124, and/or a computer-assisted surgery application 1160 may be stored in one or more data sources 1130a-n accessible to computing device 1110 via network 1190.

FIGS. 13A-C illustrate various views of an example of a tracking patella clamp with a patella preparation element in accordance with one or more features of the present disclosure. As shown in FIG. 13A, a patella tracking clamp 100 may be coupled to or otherwise associated with one or more patella preparation elements 860. In any preceding or subsequent example, a patella preparation element 860 may include a tool or other device configured to prepare the patella, such as a cutting tool, a reaming tool, a burring tool, or other tool.

In the example of FIGS. 13A-C, patella preparation element 860 is a reamer tool. In any preceding or subsequent example, reamer tool 860 may be detected by surgery system 950, for instance, via tracking system 940. For example, reamer tool 860 may have elements or properties that may be detected by tracking system 940. Referring to FIG. 13B, reamer tool 860 may include a reflective surface 861 that may be detected by a tracking system 940, such as through reflection of light (for instance, IR light) via an optical tracking system 940. In reference to FIG. 13B, reamer tool 860 may include trackers 862, for instance, on surface 861. Reflective surface 861 and/or trackers 862 may be used to determine operational characteristics of reamer tool 860. For instance, tracking system 940 may be configured to detect a location (for instance, relative to the patella) of reamer tool 860 for determining the reaming depth in a patella during a surgical procedure.

In any preceding or subsequent example, characteristics of reamer tool 860 (e.g., location, depth in patella, orientation, and/or the like) and/or a visual representation of reamer tool 860 (e.g., a “tool model”) may be presented to a viewer, such as via GUI interface 1205. In this manner, a surgeon or other operator may be able to visualize the operation of reamer tool 860 in real-time or substantially real-time, including from various angles, orientations, etc. via modeling according to any preceding or subsequent example that are not available via conventional techniques. Accordingly, using devices according to any preceding or subsequent example, a surgeon may be able to visualize and, therefore, have improved confidence in the results of reaming the patella using a computer-assisted and/or robotic surgical system.

In any preceding or subsequent example, a patella tracking clamp, such as clamp 100, may allow a surgeon or other user to determine and/or define a plane (e.g., planes 801 and 802) and allow for proper placement of a cutting tool (e.g., reamer tool 860) with precision and confidence for the surgeon. For instance, a surgeon, via tracking elements of clamp 100 and GUI interface 1205, may be able to determine if reamer tool 860 is sufficiently perpendicular to a subject cutting plane, for instance, to determine where reamer tool 860 is going to engage or “land” on the patella to ensure proper reaming.

Although a reamer tool has been used as an example of a patella preparation element in the configurations depicted in FIGS. 13A-C, any preceding or subsequent examples are not so limited. For example, cutting tools, saws, burring tools, resection guides and/or other tools, may be used in accordance with features of the present disclosure, alone or in combination with reflective surfaces 861 and/or trackers 862 to determine operational characteristics thereof. For example, a burring tool may be associated with trackers 862 to provide burring locations and depths for use by tracking system 940 and, ultimately, surgery system 950 to generate patella models.

It should be appreciated that while the feature of incorporating one or more navigation elements, and their corresponding use, will be shown and described in connection with patella clamp 100, the present disclosure should not be so limited and incorporating the navigation elements, and their corresponding use, may be used in connection with any other patella clamp now known or hereafter developed.

The foregoing description has broad application. While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the invention are to be considered within the scope of the disclosure. The present disclosure should be given the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art. Unless otherwise defined, all technical terms used herein have the same meaning as commonly understood by one of ordinary skill in the art to which the disclosure belongs.

Directional terms such as top, bottom, superior, inferior, medial, lateral, anterior, posterior, proximal, distal, upper, lower, upward, downward, left, right, longitudinal, front, back, above, below, vertical, horizontal, radial, axial, clockwise, and counter-clockwise) and the like may have been used herein. Such directional references are only used for identification purposes to aid the reader's understanding of the present disclosure. For example, the term “distal” may refer to the end farthest away from the medical professional/operator when introducing a device into a patient, while the term “proximal” may refer to the end closest to the medical professional when introducing a device into a patient. Such directional references do not necessarily create limitations, particularly as to the position, orientation, or use of this disclosure. As such, directional references should not be limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to various examples of similar sections, portions, or components herein may be interchangeably applied to any other similar example or feature shown or described herein.

It should be understood that, as described herein, an “example” or “embodiment” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated examples are to be understood as instances, configurations, specimens, embodiments, illustrations, models, examples, and/or the like (unless otherwise stated), and other manners of embodying the described concepts or features, such as may be understood by one of ordinary skill in the art upon learning the concepts or features from the present disclosure, are within the scope of the disclosure. Furthermore, references to “one embodiment,” “one example,” “preceding or subsequent examples” of the present disclosure are not intended to be interpreted as excluding the existence of additional embodiments or examples that also incorporate the recited features.

In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited. It will be further understood that the terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

The phrases “at least one”, “one or more”, and “and/or”, as used herein, are open-ended expressions that are both conjunctive and disjunctive in operation. The terms “a” (or “an”), “one or more” and “at least one” can be used interchangeably herein.

Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

1. A tracking patella clamp, comprising:
a clamping assembly operative to engage a patella;
a handle assembly coupled to the clamping assembly, the handle assembly operative to actuate to secure the patella within the clamping assembly; and
at least one tracking element operative to determine tracking information of the patella.

2. The tracking patella clamp of claim 1, wherein the handle assembly is operative to rotate relative to the clamping assembly.

3. The tracking patella clamp of claim 1, wherein the at least one tracking element is coupled to the clamping assembly.

4. The tracking patella clamp of claim 1, wherein the tracking information comprises at least one of position information or orientation information of the patella.

5. The tracking patella clamp of claim 1, wherein the tracking information comprises a cutting plane associated with the patella.

6. The tracking patella clamp of claim 1, wherein the at least one tracking element is configured to determine instrument information of a surgical instrument associated with the tracking patella clamp.

7. The tracking patella clamp of claim 6, wherein the instrument information comprises a location of the surgical instrument relative to the patella.

8. The tracking patella clamp of claim 6, wherein the at least one tracking element is configured to determine the instrument information via detecting a reflective surface of the surgical instrument.

9. The tracking patella clamp of claim 7, wherein the surgical instrument comprises a reamer tool.

10. A computer-assisted surgical system, comprising:
at least one computing device, comprising:
a display device;
processing circuitry; and
a memory coupled to the processing circuitry, the memory comprising instructions that, when executed by the processing circuitry, cause the processing circuitry to:
receive tracking information associated with a patella detected by at least one tracking element coupled to a tracking patella clamp engaging the patella,
determine patella information based on the tracking information, the patella information indicating at least one characteristic of the patella,
generate a patella model configured as a virtual representation of the patella configured based on the patella information, and
present a graphical user interface (GUI) object visually depicting the patella model on a GUI screen via the display device.

11. The computer-assisted surgical system of claim 10, the instructions, when executed by the processing circuitry, to cause the processing circuitry to receive instrument tracking information corresponding to a surgical instrument associated with the tracking patella clamp, the instrument tracking information detected by the at least one tracking element.

12. The computer-assisted surgical system of claim 11, the instructions, when executed by the processing circuitry, to cause the processing circuitry to determine instrument information of the surgical instrument based on the instrument tracking information, the instrument information comprising at least one of a position or an orientation of the surgical instrument.

13. The computer-assisted surgical system of claim 10, the instructions, when executed by the processing circuitry, to cause the processing circuitry to present a GUI model of the surgical instrument on the GUI screen based on the instrument information.

14. The computer-assisted surgical system of claim 11, wherein the surgical instrument comprises a reamer tool.

15. The computer-assisted surgical system of claim 10, wherein the tracking information comprises a cutting plane associated with the patella.
